(19) United States
(12) Patent Application Publication
(10) Pub. No.: US 2012/0029083 A1
(43) Pub. Date: Feb. 2, 2012

(54) POLYMORPHIC FORMS OF ALISKIREN HEMIFUMARATE AND PROCESS FOR PREPARATION THEREOF

(75) Inventors: Nina Finkelstein, Herzliya (IL); Boris Finkelstein, Herzliya (IL); Ariel Mittelman, Elad (IL); Tamás Koltai, Netanya (IL)

(73) Assignee: Teva Pharmaceutical Industries, Ltd.

(21) Appl. No.: 12/742,856

(22) PCT Filed: Nov. 13, 2008
(86) PCT No.: PCT/US2008/012816

§ 371 Date: Oct. 4, 2011

Related U.S. Application Data
(60) Provisional application No. 60/987,679, filed on Nov. 13, 2007.
(60) Provisional application No. 61/004,382, filed on Nov. 26, 2007.
(60) Provisional application No. 61/016,650, filed on Dec. 26, 2007.
(60) Provisional application No. 61/020,845, filed on Jan. 14, 2008.
(60) Provisional application No. 61/029,752, filed on Feb. 19, 2008.
(60) Provisional application No. 61/031,069, filed on Feb. 25, 2008.
(60) Provisional application No. 61/034,229, filed on Mar. 6, 2008.
(60) Provisional application No. 61/059,662, filed on Jun. 6, 2008.
(60) Provisional application No. 61/091,635, filed on Aug. 25, 2008.

(51) Int. Cl. A61K 31/165; C07C 237/52; A61P 9/12; C07C 231/00

(52) U.S. Cl. 514/616; 564/157

(57) ABSTRACT

Provided are amorphous and polymorphic forms of aliskiren hemifumarate, pharmaceutical compositions thereof, and processes for their preparation.

### CROSS REFERENCE TO RELATED APPLICATIONS

This application claims the benefit of U.S. Provisional Application Nos. 60/987,679 filed Nov. 13, 2007; 61/004,382 filed Nov. 26, 2007; 61/016,650 filed Dec. 26, 2007; 61/020,845 filed Jan. 14, 2008; 61/029,752 filed Feb. 19, 2008; 61/031,069 filed Feb. 25, 2008; 61/034,229 filed Mar. 6, 2008; 61/059,662 filed on Jun. 6, 2008 and 61/091,635 filed Aug. 25, 2008; hereby incorporated by reference.

### FIELD OF THE INVENTION

The present invention relates to polymorphic forms of aliskiren hemifumarate, and processes for preparing said forms.

### BACKGROUND OF THE INVENTION

Aliskiren hemifumarate [CAS Registry Number: 173334-58-2], having the chemical name: (2S,4S,5S,7S)-N-(2-Carbamoyl-2-methylpropyl)-5-amino-4-hydroxy-2,7-diisopropyl-8-[4-methoxy-3-(3-methoxypropoxy)phenyl]-octanamide hemifumarate [C₃₀H₅₃N₃O₆.0.5 C₄H₄O₄] and the following structure:

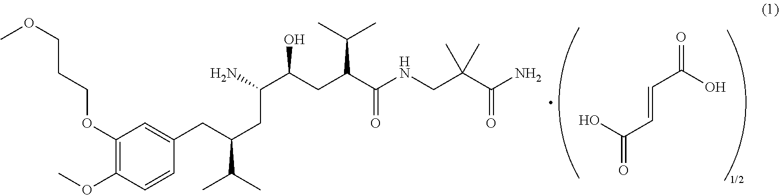

is indicated for treatment of hypertension, acting as a renin inhibitor, and marketed by Novartis as Tekturna® as a once-daily formulation. Aliskiren and its related compounds are referred to in U.S. Pat. No. 5,559,111, while synthesis, pharmacological actions, pharmacokinetics and clinical studies of aliskiren and its related compounds are referred to in Lindsay, K. B. et. al, J. Org. Chem., vol. 71, pp 4766-4777 (2006) and in Drugs of the Future, Vol. 26, No. 12, pp 1139-1148 (2001).

U.S. Pat. No. 5,559,111 refers to the preparation of a crystalline form of aliskiren hemifumarate having a melting point of about 95-104° C. by crystallizing from an ethanol/acetonitrile mixture in a 1 to 19 volume ratio and then drying at 60° C.

U.S. Pat. No. 6,730,798 refers to the preparation of aliskiren hemifumarate from aliskiren base and fumaric acid in ethanol/acetonitrile.

WO 2005/089729 (“WO '729”) refers to solid oral dosage forms comprising aliskiren obtained by a process comprising; wet granulation of the API, drying the obtained granulate, mixing with an outer phase excipient and further compressing to obtain a tablet. WO '729 discusses the difficulties in formulation of aliskiren due to the needle shaped habit of its crystals. Moreover, it claims that the compression behavior of the drug substance is poor and therefore direct compression is a difficult option for routine production.

This patent application also refers to the difficulties in formulation of aliskiren. The hurdles include the high hygroscopicity of aliskiren, its relatively low stability and the variability in drug substance quality. The latter has effect on the processability of a tablet, leading to a more complicated manufacturing process, in particular when isolating the final product.

The present invention relates to the solid state physical properties of aliskiren hemifumarate. These properties can be influenced by controlling the conditions under which aliskiren hemifumarate is obtained in solid form. Solid state physical properties include, for example, the flow-ability of the milled solid. Flow-ability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form. Thermal behavior is measured in the laboratory by such techniques as capillary melting point, thermogravimetric analysis (TGA) and differential scanning calorimetric (DSC) and can be used to distinguish some polymorphic forms from others. A particular polymorphic form may also give rise to distinct spectroscopic properties that may be detectable by powder X-ray crystallography, solid state ¹³C NMR spectrometry and infrared spectrometry.

One of the most important physical properties of a pharmaceutical compound, which can form polymorphs or solvates, is its solubility in aqueous solution, particularly the solubility in gastric juices of a patient. Other important properties relate to the ease of processing the form into pharmaceutical dosages, as the tendency of a powdered or granulated form to flow and the surface properties that determine whether crystals of the form will adhere to each other when compacted into a tablet.

The discovery of new polymorphic forms of aliskiren hemifumarate provides a new opportunity to improve the performance of the synthesis of the active pharmaceutical ingredient (API), aliskiren hemifumarate, by producing polymorphs of aliskiren hemifumarate having improved characteristics, such as flowability, and solubility. Thus, there is a need in the art for polymorphic forms of aliskiren hemifumarate.

### SUMMARY OF THE INVENTION

The invention encompasses amorphous and polymorphic forms of aliskiren hemifumarate and processes for their preparation.

In one embodiment, the present invention encompasses a crystalline form of Aliskiren hemifumarate (designated Form I), characterized by data selected from a group consisting of: a powder XRD pattern with peaks at about 3.8, 6.6, 7.6, 8.0, 13.8, 14.5, 15.6 and 17.4±0.2 degrees 2-theta, a powder XRD pattern with peaks at about 3.8, 7.6, 8.0, 13.8 and 15.6±0.2 degrees 2-theta with optional one or more additional peaks in the powder XRD pattern at about 6.6, 14.5 and 17.4±0.2 degrees 2-theta, a powder XRD pattern as depicted in FIG. 1 and combinations thereof.

In one embodiment, the invention encompasses crystalline aliskiren hemifumarate (designated Form II) characterized by a powder XRD pattern with peaks at about 4.9, 7.3, 10.0 and 12.2±0.2 degrees 2-theta. This crystalline aliskiren hemifumarate Form II may be further characterized by one or more additional peaks in the powder XRD pattern at about 8.5, 9.5, 11.8 or 21.4±0.2 degrees 2-theta or a powder XRD pattern as depicted in FIG. 2.

In another embodiment, the invention encompasses crystalline aliskiren hemifumarate (designated Form III) characterized by a powder XRD pattern with peaks at about 6.5, 7.4, 19.5 and 20.6±0.2 degrees 2-theta. This crystalline aliskiren hemifumarate Form III may be further characterized by one or more additional peaks in the powder XRD pattern at about 18.4 or 22.7±0.2 degrees 2-theta or a powder XRD pattern as depicted in FIG. 3

In yet another embodiment, the invention encompasses crystalline aliskiren hemifumarate (designated Form V) characterized by a powder XRD pattern with peaks at about 4.5, 7.0, 13.6 and 19.6±0.2 degrees 2-theta. This crystalline aliskiren hemifumarate Form V may be further characterized by one or more additional peaks in the powder XRD pattern at about 5.8, 18.2 or 22.8±0.2 degrees 2-theta or a powder XRD pattern as depicted in FIG. 4

In one embodiment, the invention encompasses crystalline aliskiren hemifumarate (designated Form VII) characterized by a powder XRD pattern with peaks at about 4.3, 6.2, 12.4 and 18.8±0.2 degrees 2-theta, designated Form VII. This crystalline aliskiren hemifumarate may be further characterized by one or more additional peaks in the powder XRD pattern at about 8.9, 10.0, 17.9 or 19.5±0.2 degrees 2-theta or a powder XRD pattern as depicted in FIG. 5.

In one embodiment, the invention encompasses crystalline aliskiren hemifumarate (designated Form VIII) characterized by a powder XRD pattern with peaks at about 6.0, 7.4, 9.3 and 11.1±0.2 degrees 2-theta. This crystalline aliskiren hemifumarate Form VIII may be further characterized by one or more additional peaks in the powder XRD pattern at about 8.6, 10.0, 19.2, 19.7 or 20.1±0.2 degrees 2-theta or a powder XRD pattern as depicted in FIG. 6.

In another embodiment, the invention encompasses crystalline aliskiren hemifumarate (designated Form IX) characterized by a powder XRD pattern with peaks at about 3.6, 6.5, 7.2, 12.4 and 18.0±0.2±0.2 degrees 2-theta. This crystalline aliskiren hemifumarate Form IX may be further characterized by one or more additional peaks in the powder XRD pattern at about 6.2, 8.5, 13.5, 18.9 or 22.3±0.2 degrees 2-theta or a powder XRD pattern as depicted in FIG. 7.

In yet another embodiment, the invention encompasses crystalline aliskiren hemifumarate (designated Form X) characterized by a powder XRD pattern with peaks at about 4.7, 6.3, 10.5 and 19.2±0.2 degrees 2-theta. This crystalline aliskiren hemifumarate Form X may be further characterized by one or more additional peaks in the powder XRD pattern at about 8.4, 9.6, 16.9, 19.5 or 24.2±0.2 degrees 2-theta or a powder XRD pattern as depicted in FIG. 8.

In one embodiment, the present invention encompasses amorphous aliskiren hemifumarate, as depicted in FIGS. 9, 10 and 11.

In one embodiment, the present invention encompasses processes for the preparation of crystalline aliskiren hemifumarate Forms I, II, III, V, VII and VIII.

In yet another embodiment, the present invention encompasses processes for the preparation of the amorphous form of aliskiren hemifumarate.

### BRIEF DESCRIPTION OF THE FIGURES

FIG. 1 represents a powder XRD pattern of crystalline aliskiren hemifumarate Form I.

FIG. 2 represents a powder XRD pattern of crystalline aliskiren hemifumarate Form II.

FIG. 3 represents a powder XRD pattern of crystalline aliskiren hemifumarate Form III.

FIG. 4 represents a powder XRD pattern of crystalline aliskiren hemifumarate Form V.

FIG. 5 represents a powder XRD pattern of crystalline aliskiren hemifumarate Form VII.

FIG. 6 represents a powder XRD pattern of crystalline aliskiren hemifumarate Form VIII.

FIG. 7 represents a powder XRD pattern of crystalline aliskiren hemifumarate Form IX.

FIG. 8 represents a powder XRD pattern of crystalline aliskiren hemifumarate Form X.

FIG. 9 represents a powder XRD pattern of amorphous aliskiren hemifumarate.

FIG. 10 represents a powder XRD pattern of amorphous aliskiren hemifumarate, prepared according to example 51

FIG. 11 represents a powder XRD pattern of amorphous aliskiren hemifumarate, prepared according to example 66

FIG. 12 represents an exemplary powder XRD pattern of crystalline aliskiren hemifumarate Form I, prepared according to dried examples 17B and 17G.

FIG. 13 represents an exemplary powder XRD pattern of crystalline aliskiren hemifumarate Form I, prepared according to dried examples 17C, 17D, 17E and 17H.

FIG. 14 represents a powder XRD pattern comparison of a) the starting amorphous aliskiren hemifumarate (top), b) the placebo (middle), and c) the formulated tablet of amorphous aliskiren hemifumarate (bottom).

### DETAILED DESCRIPTION OF THE INVENTION

The invention addresses a need in the art by providing crystalline forms of aliskiren hemifumarate and processes for their preparation.

As used herein, a “sufficient” period necessary to obtain a desired polymorphic form can be determined by periodically measuring a sample by powder XRD until the desired polymorphic form is obtained.

As used herein, “fast evaporation” refers to dissolving aliskiren hemifumarate in a solvent and fast-removing the solvent under reduced pressure. Preferably, reduced pressure is below a pressure of 760 mmHg or 1 atmosphere.

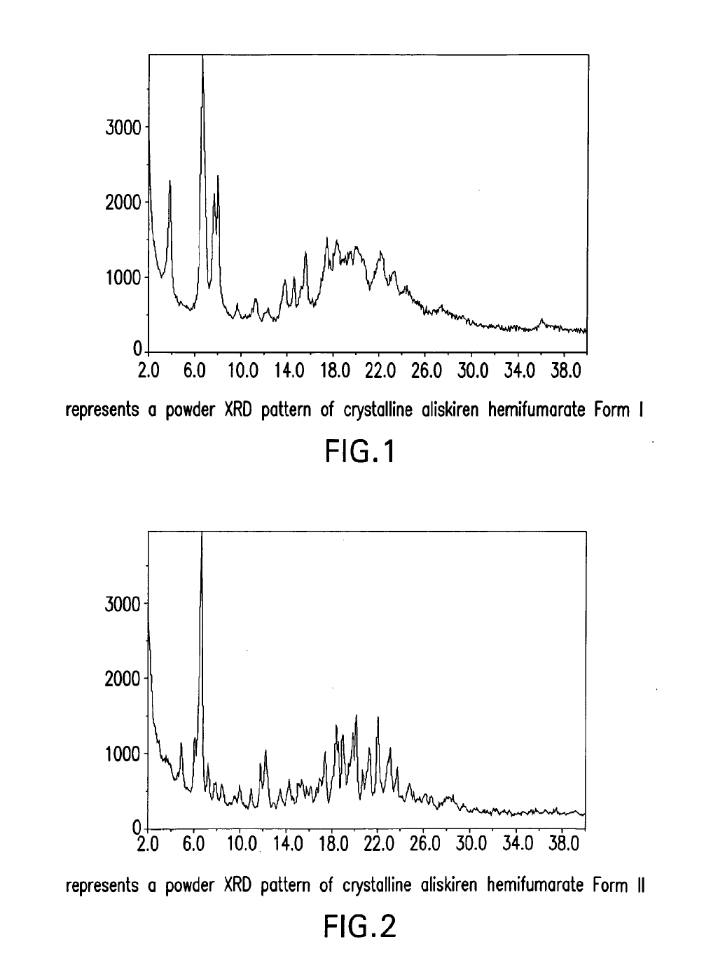

In one embodiment, the present invention encompasses a crystalline form of Aliskiren hemifumarate (designated Form I), characterized by data selected from a group consisting of: a powder XRD pattern with peaks at about 3.8, 6.6, 7.6, 8.0, 13.8, 14.5, 15.6 and 17.4±0.2 degrees 2-theta, a powder XRD pattern with peaks at about 3.8, 7.6, 8.0, 13.8 and 15.6±0.2 degrees 2-theta with optional one or more additional peaks in the powder XRD pattern at about 6.6, 14.5 and 17.4±0.2 degrees 2-theta, a powder XRD pattern as depicted in FIG. 1 and combination thereof.

Aliskiren hemifumarate Form I may be prepared by a number of methods.

In one example, aliskiren hemifumarate Form I is crystallized from ethanol and acetonitrile. Preferably, the aliskiren hemifumarate/ethanol ratio used in the above process is 1:1 of g aliskiren hemifumarate to g ethanol and the acetonitrile/ethanol volume ratio is 18:1 (v/v). Preferably, the crystallization is performed by providing a solution of aliskiren hemifumarate in ethanol and admixing the solution with an antisolvent such as acetonitrile to crystallize aliskiren hemifumarate Form I. The solution of aliskiren hemifumarate may be prepared, for example, by mixing aliskiren, fumaric acid and ethanol. Crystallization can be carried out without seeding.

In another example, aliskiren hemifumarate is crystallized from a solvent selected from the group consisting of diethylcarbonate, tetrahydrofuran and ethyl acetate. With ethyl acetate a ratio of lower than about 1:40 g of aliskiren hemifumarate to ml ethyl acetate is used (w/v). Preferably, a ratio of about 1:15 to about 1:25 is used. Preferably, the crystallization is performed by dissolving aliskiren hemifumarate in the solvent, and cooling the solution to crystallize the aliskiren hemifumarate Form I. Aliskiren hemifumarate may be formed in situ by combining aliskiren base and fumaric acid. The aliskiren hemifumarate may be dissolved in the solvent at any temperature between about 25° C. and about 100° C., preferably by gradual heating to a temperature between about 25° C. and about 100° C. The resulting solution may be cooled at a temperature of about −10° C. to about 20° C. to crystallize the aliskiren hemifumarate Form I.

In another example, Form I is crystallized from an alcohol selected from the group consisting of ethanol and 1-propanol and an antisolvent selected from the group consisting of isopropyl acetate, ethyl acetate, butyl acetate, isobutyl acetate, methyl tert-butyl ether. Preferably, the crystallization is performed by providing a solution of aliskiren hemifumarate in alcohol and admixing the solution with an antisolvent selected from a group consisting of isopropyl acetate, ethyl acetate, butyl acetate, isobutyl acetate, methyl tert-butyl ether to crystallize aliskiren hemifumarate Form I. Preferably, the antisolvent is present in an amount of about 5 ml to 25 ml per gram of aliskiren hemifumarate, and more preferably about 5 to 15 ml per gram of aliskiren hemifumarate. With butyl-acetate, preferably a ratio of higher than about 1:15 g aliskiren hemufumarate to ml butyl acetate is used (w/v). Preferably, the ratio is about 1:20 to about 1:30. With ethyl acetate, preferably a ratio of lower than about 1:27 g aliskiren hemufumarate to ml ethyl acetate is used. Preferably, the ratio is about 1:15 to about 1:25.

In another embodiment, heptane or acetonitrile may be used as an antisolvent when ethanol is used as an alcohol in the process described above.

In another example, Form I is prepared by drying aliskiren hemifumarate Form II, III, IX or X, described below, or a mixture thereof at about 25° C. to about 70° C., to obtain aliskiren hemifumarate Form I. Preferably, the drying is performed at a temperature of about 40° C. to about 60° C. Preferably, the drying is performed under vacuum (pressure of less than 100 mmHg). Drying can be performed for at least about 6 hours, and such as about 12 hours.

In one embodiment, the invention encompasses aliskiren hemifumarate Form I, prepared according to any of the processes described above.

In one embodiment, the invention encompasses crystalline aliskiren hemifumarate (designated Form II) characterized by a powder XRD pattern with peaks at about 4.9, 7.3, 10.0 and 12.2±0.2 degrees 2-theta. This crystalline aliskiren hemifumarate Form II may be further characterized by one or more additional peaks in the powder XRD pattern at about 8.5, 9.5, 11.8 or 21.4±0.2 degrees 2-theta or a powder XRD pattern as depicted in FIG. 2. Crystalline aliskiren hemifumarate Form II may also be characterized by a powder XRD pattern with peaks at about 4.9, 7.3, 10.0 and 12.2±0.2 and an absence of peak at about 9.2 degrees 2-theta.

Aliskiren hemifumarate Form II may be prepared by a number of methods.

In one example, aliskiren hemifumarate Form II is crystallized from a solvent selected from the group consisting of iso-butyl acetate and butyl acetate. Preferably, the crystallization is performed by dissolving aliskiren hemifumarate in the solvent, and then cooling the solution to crystallize the aliskiren hemifumarate Form II. Preferably, the aliskiren hemifumarate is dissolved in the solvent by heating a mixture of the aliskiren hemifumarate and the solvent, preferably by gradual heating to a temperature between 25° C. and 100° C. Preferably, the solution is cooled at a temperature of about −10° C. to about 20° C. to crystallize the aliskiren hemifumarate Form II.

In another example, aliskiren hemifumarate Form II is prepared by a process comprising suspending (heterogeneous mixture) aliskiren hemifumarate Form I in a solvent to obtain aliskiren hemifumarate Form II, and recovering the aliskiren hemifumarate Form II from the suspension, wherein the solvent is selected from the group consisting of: 2-pentanol, methyl tert-butyl ether, diethylcarbonate, butyl acetate, isobutyl acetate, ethanol, toluene, mixtures thereof, and mixtures thereof with water. Preferably, the suspension is maintained at about room temperature for a period of about 10 to about 40 hours. The solvent-water mixture preferably contains about 0.3% to about 0.9% water.

In yet another example, aliskiren hemifumarate is crystallized from ethanol with the aid of an antisolvent. Preferably, the crystallization is performed by providing a solution of aliskiren hemifumarate in ethanol and admixing the solution with an antisolvent selected from the group consisting of diethylcarbonate, ethyl acetate, and butyl acetate to crystallize aliskiren hemifumarate Form II. With butyl-acetate, preferably a ratio lower than about 1:15 g aliskiren hemufumarate to ml butyl acetate (w/v) is used and the solvent to anti-solvent volume ratio is about 1:3 to about 1:8 (v/v). With ethyl acetate, preferably a ratio greater than about 1:27 g aliskiren hemufumarate to ml ethyl acetate is used and the solvent to anti-solvent volume ratio is about 1:12 to about 1:18 (v/v).

In another example, amorphous aliskiren hemifumarate is combined with dimethyl carbonate, isobutyl acetate for a period sufficient to allow conversion to aliskiren hemifumarate Form II. Preferably, the suspension is maintained for a period of about 25 to about 45 hours. More preferably, for a period of about 30 to about 40 hours.

In yet another example, a mixture of aliskiren base, fumaric acid and iso-butyl acetate or heptane-ethanol is maintained for a period sufficient to obtain Form II. Preferably, the suspension is maintained for a period of about 10 to about 30 hours. More preferably, for a period of about 20 to about 25 hours.

In yet another example, aliskiren hemifumarate Form I is exposed to methyl tert-butyl ether or n-butyl acetate for a period sufficient to obtain Form II. Preferably, the suspension is maintained for a period of about 24 hours to about 50 days. More preferably, for a period of about 30 to about 40 hours.

In yet another example, wet granulation of Form I in butyl acetate or ethanol or water-iso-propanol results in Form II. As used herein, the term “wet granulation” refers to a process whereby the aliskiren is mixed with a minimal amount of solvent, and stirring the mixture at room temperature for the time needed to cause the desired transformation. This can be done with a mechanical stirrer or rotary evaporator. About 0.1 ml to about 0.2 ml of solvent per gram of the compound is preferably used. For small quantities, an evaporator without vacuum can be used.

In one embodiment, the invention encompasses aliskiren hemifumarate Form II, prepared according to any of the processes described above.

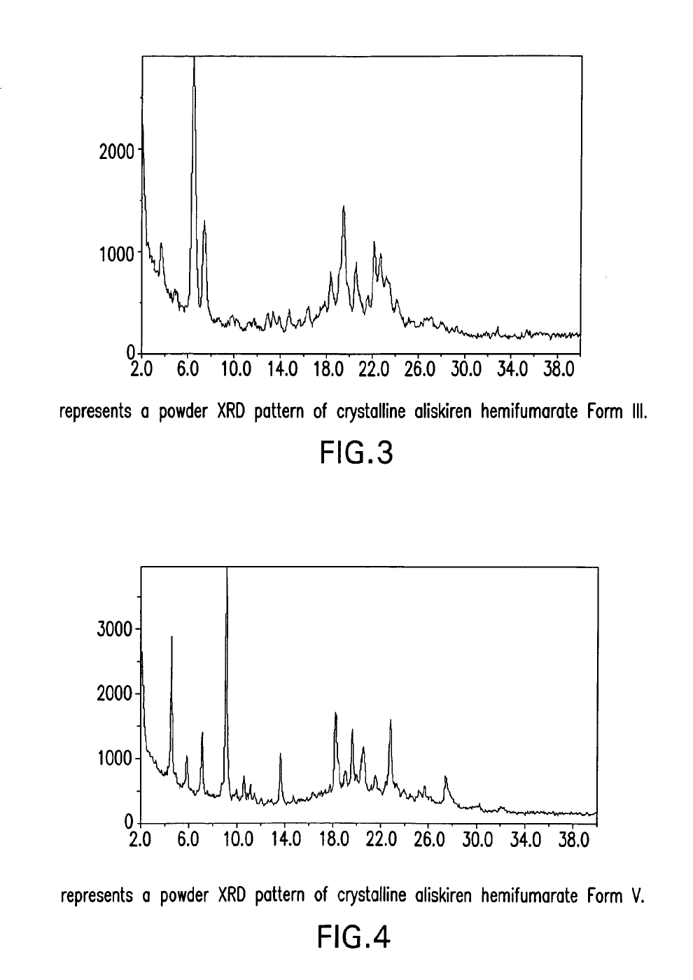

In another embodiment, the invention encompasses crystalline aliskiren hemifumarate (designated Form III) characterized by a powder XRD pattern with peaks at about 6.5, 7.4, 19.5 and 20.6±0.2 degrees 2-theta. This crystalline aliskiren hemifumarate Form III may be further characterized by one or more additional peaks in the powder XRD pattern at about 18.4 or 22.7±0.2 degrees 2-theta or a powder XRD pattern as depicted in FIG. 3. Crystalline aliskiren hemifumarate Form III may also be characterized by a powder XRD pattern with peaks at about 6.5, 7.4, 19.5 and 20.6±0.2 and an absence of peak at about 8.0 degrees 2-theta.

The aliskiren hemifumarate Form III may be prepared by a process comprising suspending aliskiren hemifumarate Form I in a solvent to obtain aliskiren hemifumarate Form III, and recovering aliskiren hemifumarate Form III from the suspension, wherein the solvent is selected from the group consisting of 2-butanol, methyl acetate. Recovery can be carried out with filtration. Preferably, the suspension is maintained at about room temperature for a period of time sufficient to obtain the aliskiren hemifumarate Form III. More preferably, the suspension is maintained for a period of about 30 to about 40 hours. The ratio of aliskiren hemifumarate to the solvent is preferably about 1:5 to about 1:15 of g aliskiren hemifumarate to ml solvent. More preferably, the ratio is 1:10.

In another embodiment, aliskiren hemifumarate Form III may be prepared according to the process described wherein the solvent is tetrahydrofuran.

In one embodiment, the invention encompasses aliskiren hemifumarate Form III, prepared according to any of the processes described above.

In another embodiment, the invention encompasses crystalline aliskiren hemifumarate (designated Form V) characterized by a powder XRD pattern with peaks at about 4.5, 7.0, 13.6 and 19.6±0.2 degrees 2-theta. This crystalline aliskiren hemifumarate Form V may be further characterized by one or more additional peaks in the powder XRD pattern at about 5.8, 18.2 or 22.8±0.2 degrees 2-theta or a powder XRD pattern as depicted in FIG. 4.

The aliskiren hemifumarate Form V may be prepared by a process comprising suspending aliskiren hemifumarate (or aliskiren base and fumaric acid) in acetonitrile for a period sufficient to obtain aliskiren hemifumarate Form V, and recovering aliskiren hemifumarate Form V from the suspension. The aliskiren hemifumarate in acetonitrile may be in solution form or aliskiren hemifumarate amorphous or Form I in suspension with acetonitrile. When aliskiren hemifumarate Form I is used, the ratio of aliskiren hemufumarate to acetonitrile is preferably lower than about 1:15 g of aliskiren hemifumarate to ml of acetonitrile (w/v). Preferably, the suspension is maintained for a period of about 25 to about 45 hours. More preferably, the suspension is maintained for a period of about 30 to about 40 hours. Preferably, when Form I is used, it is dissolved in acetonitrile by heating a mixture of Form I and acetonitrile, preferably by gradual heating to a temperature between 25° C. and 100° C. Preferably, the solution is cooled at a temperature of about −10° C. to about 20° C. to crystallize the aliskiren hemifumarate Form V. Recovery can be carried out by filtration. The ratio of aliskiren hemifumarate to the solvent is preferably about 1:5 to about 1:25 of g aliskiren hemifumarate to ml solvent (w/v). More preferably, the ratio is 1:10 to about 1:20.

The aliskiren hemifumarate Form V may also be prepared by a process comprising: dissolving aliskiren hemifumarate in ethanol, adding acetonitrile, heating the mixture to reflux and further cooling. Preferably, cooling is at a room temperature and for a period of time sufficient to obtain aliskiren hemifumarate Form V. Preferably, the solution is maintained for a period of about 24 hours to about 5 days. More preferably, for a period of about 2 to about 3 days.

In one embodiment, the invention encompasses aliskiren hemifumarate Form V, prepared according to any of the processes described above.

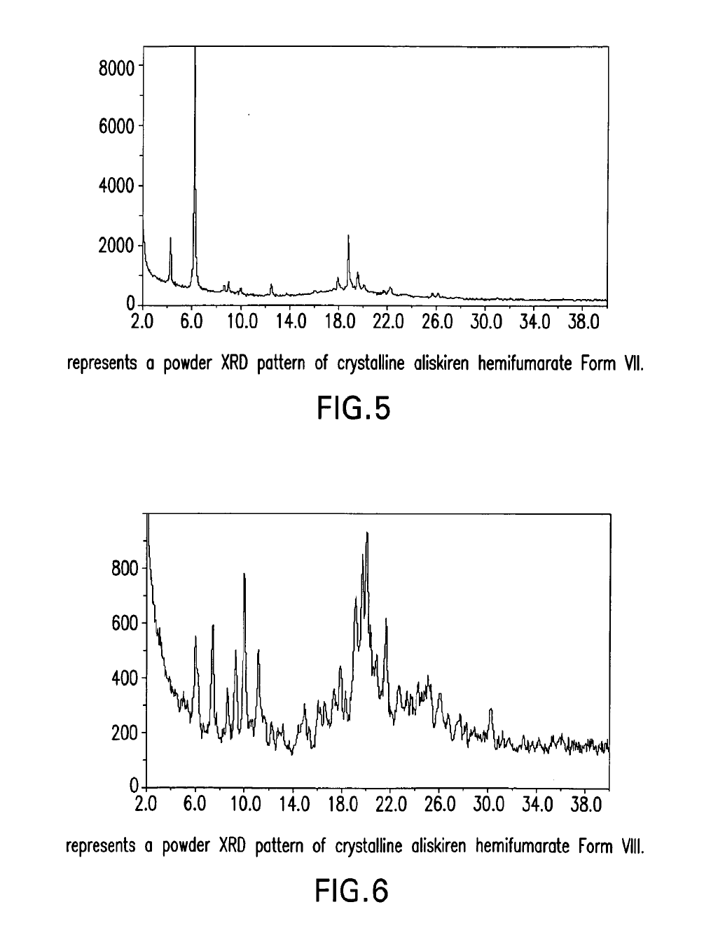

In another embodiment, the invention encompasses crystalline aliskiren hemifumarate (designated Form VII) characterized by a powder XRD pattern with peaks at about 4.3, 6.2, 12.4 and 18.8±0.2 degrees 2-theta, designated Form VII. This crystalline aliskiren hemifumarate may be further characterized by one or more additional peaks in the powder XRD pattern at about 8.9, 10.0, 17.9 or 19.5±0.2 degrees 2-theta or a powder XRD pattern as depicted in FIG. 5. Crystalline aliskiren hemifumarate Form VII may also be characterized by a powder XRD pattern with peaks at about 4.3, 6.2, 12.4 and 18.8±0.2 and an absence of peak at about 7.1 degrees 2-theta.

The aliskiren hemifumarate Form VII may be prepared by a process comprising dissolving aliskiren hemifumarate in tert-butanol, preferably by gradual heating to a temperature between 25° C. and 100° C.; and crystallizing aliskiren hemifumarate Form VII from the solution, by a method such as by cooling. Preferably, cooling is at a temperature of about −10° C. to about 20° C.

In one embodiment, the invention encompasses aliskiren hemifumarate Form VII, prepared according to any of the processes described above.

In another embodiment, the invention encompasses crystalline aliskiren hemifumarate (designated Form VIII) characterized by a powder XRD pattern with peaks at about 6.0, 7.4, 9.3 and 11.1±0.2 degrees 2-theta. This crystalline aliskiren hemifumarate Form VIII may be further characterized by one or more additional peaks in the powder XRD pattern at about 8.6, 10.0, 19.2, 19.7 or 20.1±0.2 degrees 2-theta or a powder XRD pattern as depicted in FIG. 6.

Aliskiren hemifumarate Form VIII may be prepared by a number of methods.

In one method, Form VIII is prepared by exposing aliskiren hemifumarate Form I to acetonitrile. The aliskiren hemifumarate may be exposed to atmospheric acetonitrile by maintaining an open container with aliskiren hemifumarate inside a closed container containing acetonitrile. Aliskiren hemifumarate may be exposed to acetonitrile for example when aliskiren hemifumarate is in solution with ethanol, in a suspension with acetonitrile or as crystals.

In another process, Form VIII may be prepared by drying aliskiren hemifumarate Form V for a period sufficient to allow conversion to Form VIII. Preferably, the drying is performed for a period of about 5 hours to 25 hours. More preferably, for a period of about 10 to about 15 hours. Preferably, the drying is at a temperature of about 25° C. to about 70° C., more preferably about 40° C. to about 60° C.

In yet another process, aliskiren hemifumarate Form VIII is prepared by a process comprising admixing aliskiren hemifumarate Form I or amorphous form and acetonitrile to obtain a suspension of aliskiren hemifumarate Form VIII, and recovering the aliskiren hemifumarate Form VIII from the suspension. Preferably, the aliskiren hemifumarate Form I or amorphous form is admixed in the acetonitrile at a temperature of about 20° C. to about 30° C. When aliskiren hemifumarate Form I is used, the ratio of aliskiren hemufumarate to acetonitrile is preferably greater than about 1:15 g of aliskiren hemifumarate to ml of acetonitrile (w/v).

In one embodiment, the invention encompasses aliskiren hemifumarate Form VIII, prepared according to any of the processes described above.

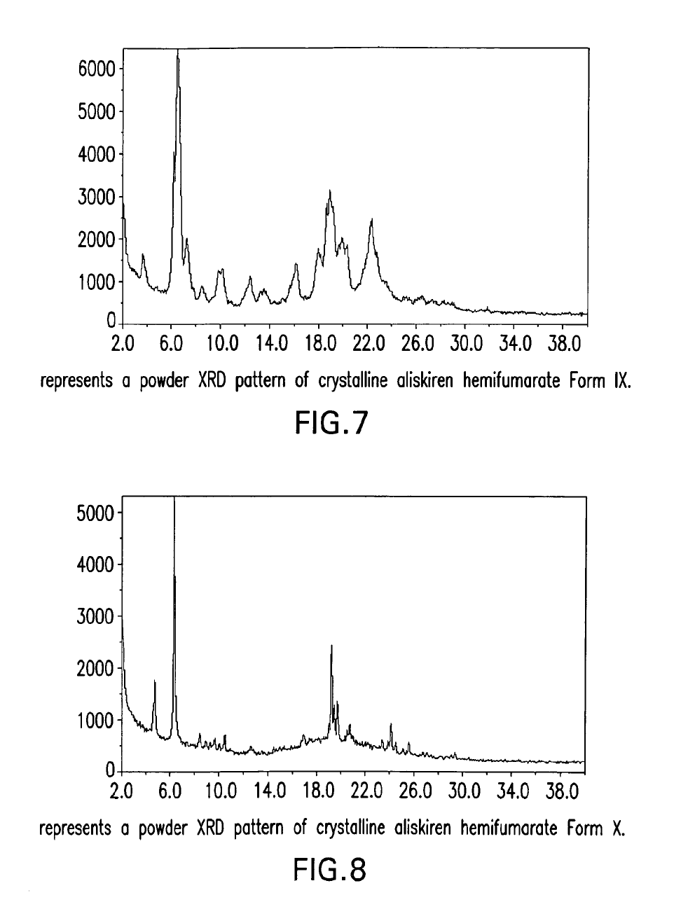

In another embodiment, the invention encompasses crystalline aliskiren hemifumarate (designated Form IX) characterized by a powder XRD pattern with peaks at about 3.6, 6.5, 7.2, 12.4 and 18.0±0.2±0.2 degrees 2-theta. This crystalline aliskiren hemifumarate Form IX may be further characterized by one or more additional peaks in the powder XRD pattern at about 6.2, 8.5, 13.5, 18.9 or 22.3±0.2 degrees 2-theta or a powder XRD pattern as depicted in FIG. 7. Crystalline aliskiren hemifumarate Form IX may also be characterized by a powder XRD pattern with peaks at about 3.6, 6.5, 7.2, 12.4 and 18.0±0.2 and an absence of peak at about 8.0 degrees 2-theta.

Aliskiren hemifumarate Form IX may be prepared by combining aliskiren base, fumaric acid with diethylcarbonate or ethyl acetate for a period of time sufficient to obtain aliskiren Form IX. Preferably, the suspension is maintained for a period of about 25 to about 45 hours. More preferably, the suspension is maintained for a period of about 30 to about 40 hours.

In another example, Form IX is crystallized from 1-propanol and methyl tert-butyl ether. Preferably, the crystallization is performed by providing a solution of aliskiren hemifumarate in 1-propanol and admixing the solution with methyl tert-butyl ether for a period of time sufficient to crystallize aliskiren hemifumarate Form IX. Preferably, the solution is maintained for a period of about 25 to about 45 hours. More preferably, for a period of about 20 to about 35 hours.

In one embodiment, the invention encompasses aliskiren hemifumarate Form IX, prepared according to any of the processes described above.

In another embodiment, the invention encompasses crystalline aliskiren hemifumarate (designated Form X) characterized by a powder XRD pattern with peaks at about 4.7, 6.3, 10.5 and 19.2±0.2 degrees 2-theta. This crystalline aliskiren hemifumarate Form X may be further characterized by one or more additional peaks in the powder XRD pattern at about 8.4, 9.6, 16.9, 19.5 or 24.2±0.2 degrees 2-theta or a powder XRD pattern as depicted in FIG. 8.

Aliskiren hemifumarate Form X may be prepared by a process comprising combining amorphous aliskiren hemifumarate with isopropanol and maintaining the combination for a period sufficient to obtain aliskiren hemifumarate Form X. Preferably, the solution is maintained for a period of about 24 hours to about 5 days. More preferably, for a period of about 2 to about 3 days.

In one embodiment, the invention encompasses aliskiren hemifumarate Form X, prepared according to any of the processes described above.

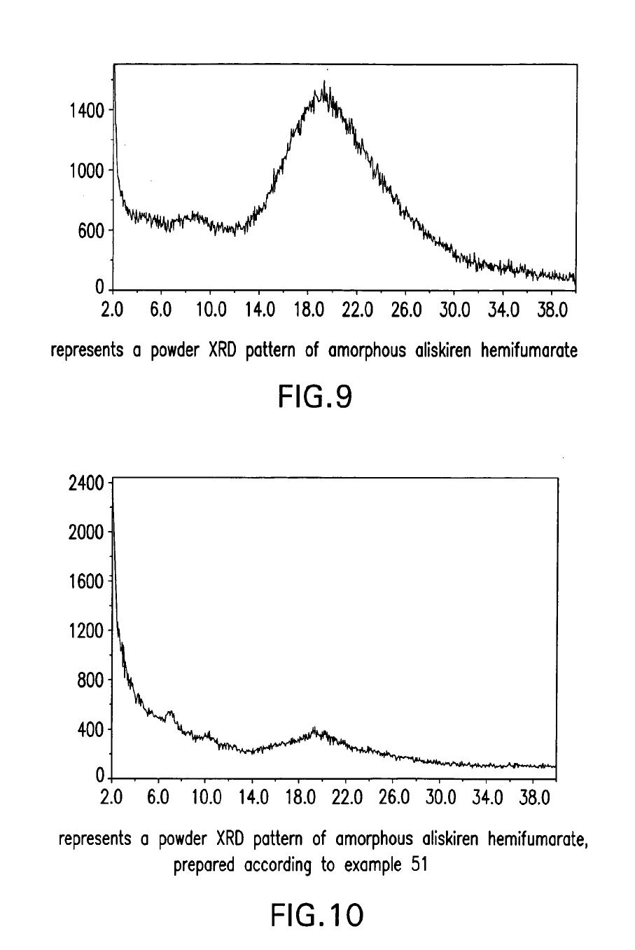

In another embodiment, the present invention encompasses amorphous aliskiren hemifumarate, as depicted in FIGS. 9, 10 and 11. Typically, the amorphous aliskiren hemifumarate has less than about 5 percent crystallinity, preferably less than about 3 percent crystallinity, and more preferably less than about 1 percent crystallinity.

The amorphous aliskiren hemifumarate described above is stable under a pressure of 2 tons for 1 minute.

In another embodiment, the present invention encompasses a number of methods for preparing the amorphous aliskiren hemifumarate.

In one method, the amorphous aliskiren hemifumarate may be prepared by a process comprising providing a solution of aliskiren hemifumarate in a solvent selected from the group consisting of butyl acetate, ethyl acetate, dimethyl carbonate, decane, dibutylether, iso-butanol and isobutyl acetate; and precipitating the amorphous aliskiren hemifumarate from the solution. The aliskiren hemifumarate in the solvent may be in solution form or aliskiren hemifumarate Form I in suspension with the solvent. With ethyl acetate, a ratio greater than about 1:40 of g aliskiren hemifumarate to 1 ml ethyl acetate is used (w/v). Preferably, a ratio of about 1:45 to about 1:65 is used. Preferably, the aliskiren hemifumarate is dissolved in the solvent by heating a mixture of the aliskiren hemifumarate and the solvent to a temperature between 25° C. and 100° C., preferably by gradual heating. Preferably, the amorphous aliskiren hemifumarate is precipitated from the solution by cooling the solution to a temperature between about 25° C. and about 0° C.

In another embodiment, the amorphous aliskiren hemifumarate may be prepared according to the process described wherein the solvent is dioxane.

In another method, amorphous aliskiren hemifumarate may be prepared by providing a solution of aliskiren hemifumarate in a C₁₋₆ alcohol such as ethanol or methanol, and removing the alcohol from the solution to obtain amorphous aliskiren hemifumarate. Preferably, the alcohol is evaporated from the solution under vacuum (pressure of less than 100 mmHg), or removed from the solution by fast evaporation. Alternatively, the amorphous aliskiren hemifumarate may be prepared by a process comprising suspending aliskiren hemifumarate Form I in a solvent to obtain amorphous aliskiren hemifumarate, and recovering amorphous aliskiren hemifumarate from the suspension, wherein the solvent is selected from the group consisting of isopropanol, heptane, iso-butanol, amyl alcohol, n-butanol, 2-butanol and tert-butanol. Preferably, the suspension is maintained at about room temperature for a period of time sufficient to obtain the amorphous aliskiren hemifumarate. Preferably, the suspension is maintained for a period of about 25 hours to about 45 hours. More preferably, for a period of about 30 to about 40 hours.

Alternatively, the amorphous aliskiren hemifumarate may be prepared by a process comprising drying of aliskiren hemifumarate Forms VII. Preferably, the aliskiren hemifumarate is dried at a temperature of about 50° C., and more preferably under vacuum.

In yet another method, amorphous aliskiren is prepared by combining aliskiren base, fumaric acid and ethanol or MTBE (methyl tert-butyl ether) for a period sufficient to obtain amorphous form. Preferably, the solution is maintained for a period of about 10 hours to about 40 hours. More preferably, for a period of about 15 to about 25 hours.

In one embodiment, the invention encompasses an amorphous aliskiren hemifumarate prepared according to any of the processes described above.

Preferably, the amorphous form described above is stable under a pressure of 2 tons for 1 minute (as depicted in FIG. 14).

The invention further encompasses a pharmaceutical formulation comprising one or more of above-described aliskiren hemifumarate Forms I, II, III, V, VII, VIII, IX, X or amorphous form. This pharmaceutical composition may additionally comprise at least one pharmaceutically acceptable excipient.

The invention further encompasses a pharmaceutical composition comprising one or more of the above-described aliskiren hemifumarate Forms I, II, III, V, VII, VIII, IX, X, or amorphous form made by the processes of the present invention, and one or more pharmaceutically acceptable excipients.

The present invention further encompasses a process for preparing a pharmaceutical formulation comprising combining one or more of the above-described aliskiren hemifumarate Forms I, II, III, V, VII, VIII, IX, X, or amorphous form, with at least one pharmaceutically acceptable excipient.

The invention further encompasses the use of one or more of the above-described aliskiren hemifumarate Forms I, II, III, V, VII, VIII, IX, X, or amorphous form, for the manufacture of a pharmaceutical composition for the treatment of hypertension, including bacterial infections, gram-negative bacterial infections, and lethal infections.

Pharmaceutical formulations of the present invention contain at least one of the above-described aliskiren hemifumarate Forms I, II, III, V, VII, VIII, IX, X, or amorphous form. In addition to the aliskiren hemifumarate, the pharmaceutical formulations of the present invention can contain one or more excipients.

The invention further encompasses methods of treating hypertension in mammals, preferably humans, by administering a therapeutically effective amount of one or more of the above-described aliskiren hemifumarate Forms I, II, III, V, VII, VIII, IX, X, or amorphous form.

Having thus described the invention with reference to particular preferred embodiments and illustrative examples, those in the art can appreciate modifications to the invention as described and illustrated that do not depart from the spirit and scope of the invention as disclosed in the specification. The examples are set forth to aid in understanding the invention but are not intended to, and should not be construed to limit its scope in any way.

### EXAMPLES

### Powder XRD (X-Ray Diffraction)

ARL X-ray powder diffractometer model X′TRA-030, Peltier detector, round standard aluminum sample holder with round zero background quartz plate (or silicon plate in the case of Form XIV) was used. The cathode is CuK_{α} radiation, λ=1.5418 Å. Scanning parameters: Range: 2-40°2θ, continuous Scan, Rate: 3 deg/min. The accuracy of peak positions is defined as +/−0.2° due to experimental differences like instrumentations, and sample preparations.

### Preparation of Aliskiren Hemifumarate Form I

### Example 1

Aliskiren base (7.13 g, 12.93 mmol) and fumaric acid (0.75 g, 6.45 mmol) were dissolved in ethanol (21 ml) at room temperature. Ethanol was evaporated from the clear solution under vacuum to total weight of 16 g, and then acetonitrile (180 ml) was added to the solution at 35° C. by portion. The solution became slurry after stirring at room temperature overnight and then cooled on ice bath and continue stirring with cooling for two hours. The solid was separated by filtration, washed with acetonitrile (2×21 ml), dried under vacuum overnight at 35° C. to obtain aliskiren hemifumarate (6.0 g).

### Example 2

Aliskiren hemifumarate (50 mg) was dissolved in diethylcarbonate (1 ml) by gradual heating (0.1-0.2° C. per minute) to 90° C. while stirring. The solution was slowly cooled to 5° C. and then heated to room temperature. A sample from the suspension was analyzed by powder XRD and found to be Form I.

### Example 3

Aliskiren hemifumarate Form I was prepared by drying of aliskiren hemifumarate Form II, Form III, Form IX or Form X (wet samples) at 50° C. under vacuum overnight.

### Example 5

Amorphous Aliskiren hemifumarate (50 mg) was dissolved in 0.5 ml of tetrahydrofuran and stirred at room temperature for 40 hours. The solution became a suspension during this time. The solid was filtered from the suspension and analyzed by powder XRD.

### Example 6

Aliskiren hemifumarate (50 mg) was dissolved in 1.0 ml ethyl acetate by gradual heating (0.1-0.2° C. per minute) to 78° C. while stirring. The solution was cooled to 5° C. and then heated to room temperature. The solid from the suspension was analyzed by powder XRD and found to be Form I.

### Example 7

Aliskiren hemifumarate (100 mg) was dissolved in 0.25 ml of alcohol. 1 ml of antisolvent was added and the solution was stirred at room temperature for 30 hours. The solid from the suspension was analyzed by powder XRD and found to be Form I. The alcohols and antisolvents used are listed in the table below.

| Alcohol | Antisolvent |
|---|---|
| Ethanol | Heptane |
| Ethanol | Isopropyl acetate |
| 1-Propanol | Ethyl acetate |
| 1-Propanol | Butyl acetate |
| 1-Propanol | Heptane |

### Example 8

Aliskiren hemifumarate (100 mg) was dissolved in 0.25 ml alcohol at room temperature. 2.5 ml antisolvent was added and the solution was stirred at room temperature for 30 hours. The solid from the suspension was analyzed by powder XRD and found to be Form I. The alcohols and antisolvents used are listed in the table below.

| Alcohol | Antisolvent |
|---|---|
| Ethanol | Isobutyl acetate |
| Ethanol | Methyl tert butyl ether |
| Ethanol | Ethyl acetate |
| Ethanol | Heptane |
| Ethanol | Butyl acetate |
| Ethanol | Isopropyl acetate |
| 1-propanol | Ethyl acetate |
| 1-propanol | Butyl acetate |
| 1-propanol | heptane |

### Preparation of Aliskiren Hemifumarate Form II

### Example 9

Aliskiren hemifumarate (50 mg) was dissolved in iso-butyl acetate (1.0 ml) by gradual heating (0.1-0.2° C. per minute) to 90° C. The solution was cooled slowly to 5° C. and then heated to room temperature. A sample from the suspension was analyzed by powder XRD and found to be Form II.

### Example 10

A suspension of aliskiren hemifumarate Form I (30 mg) was stirred in 0.3 ml of 2-pentanol at room temperature for 40 hours. The sample was filtered and analyzed by powder XRD.

### Example 11

A suspension of aliskiren hemifumarate Form I (30 mg) was stirred in 0.3 ml of methyl tert-butyl ether at room temperature for 40 hours. The sample was filtered and analyzed by powder XRD.

### Example 12

A suspension of aliskiren hemifumarate Form I (30 mg) was stirred in 0.3 ml of butyl acetate at room temperature for 40 hours. The solid was filtered and was analyzed by powder XRD. (See FIG. 2)

### Example 13

Aliskiren hemifumarate Form I (30 mg) was stirred in ethanol (0.006 ml) at room temperature on a rotor evaporator under atmospheric pressure for 10 hours. The sample was analyzed by powder XRD.

### Example 14

Aliskiren hemifumarate Form I (30 mg) was stirred in water:iso-propanol:1:1 (volume) (0.006 ml) at room temperature on a rotor evaporator under atmospheric pressure for 10 hours. The sample was analyzed by powder XRD.

### Example 15

Aliskiren hemifumarate (50 mg) was dissolved in 125 μl of ethanol. Diethylcarbonate was added to the solution in two portions (0.5 ml and then 1 ml) and the resulting suspension was stirred at room temperature for 40 hours. The solid was then filtered from the suspension and analyzed by powder XRD.

### Example 16

Aliskiren hemifumarate (50 mg) was dissolved in 125 μl of ethanol. Ethyl acetate was added to the solution in two portions (0.5 ml and then 1 ml) and the resulting suspension was stirred at room temperature for 40 hours. The solid was then filtered from the suspension and analyzed by powder XRD.

### Example 17

A suspension of aliskiren hemifumarate Form I (50 mg) was stirred in 0.5 ml of an organic solvent containing a volume percent of water as in the table below at room temperature for 20 hours. The solid was then filtered from the suspension, analyzed by powder XRD and were identified to be aliskiren hemifumarate Form II.

| | Organic Solvent | Volume % of Water |
|---|---|---|
| A | Methyl tert-butyl ether | 0.4 |
| B | Diethylcarbonate | 0.4 |
| C | Butyl acetate | 0.4 |
| D | Iso-butyl acetate | 0.4 |
| E | Butyl acetate | 0.8 |
| F | Methyl tert-butyl ether | 0.8 |
| G | Diethylcarbonate | 0.8 |
| H | Iso-butyl acetate | 0.8 |

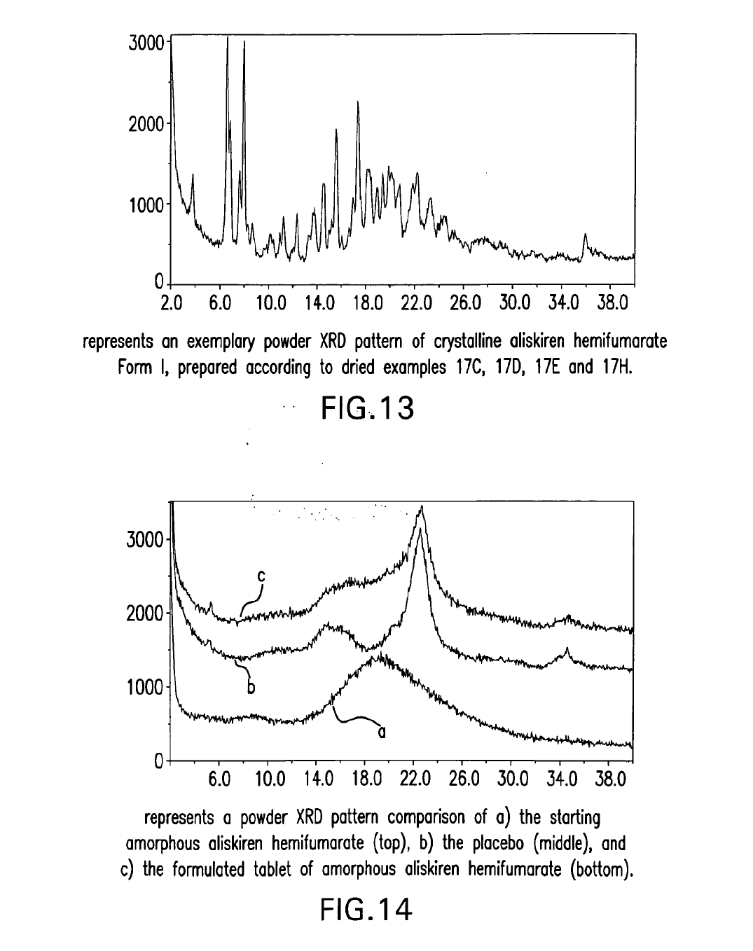

Samples A-H were also further dried to obtain aliskiren hemifumarate Form I (see FIGS. 12 and 13).

### Example 18

Aliskiren hemifumarate Form I (75 mg) was stirred in 0.75 ml of toluene at room temperature for 40 hours. A sample of the solid from the suspension was analyzed by powder XRD.

### Example 19

Aliskiren hemifumarate Form I (30 mg) was stirred in water-ethanol:1:1 (volume) (0.006 ml) on rotor evaporator at room temperature under atmospheric pressure for 10 hours to give a solution. Butyl acetate (0.5 ml) was added to the solution to precipitate aliskiren hemifumarate. A sample of the solid from the resulting suspension was analyzed by powder XRD.

### Example 20

Aliskiren hemifumarate amorphous Form (50 mg) was stirred in 0.5 ml of dimethyl carbonate at room temperature for an hour. Dimethyl carbonate (0.5 ml) was added and the suspension was stirred at room temperature for 40 hours. The solid was filtered from the suspension and analyzed by powder XRD.

### Example 21

Aliskiren hemifumarate amorphous Form (50 mg) was stirred in 0.5 ml of iso-butyl acetate at room temperature for 40 hours. The solid was filtered from the suspension and analyzed by powder XRD.

### Example 22

Aliskiren base (50 mg) was dissolved in 0.5 ml of iso-butyl acetate, and fumaric acid (5.3 mg) was added to the solution. The resulting suspension was stirred at room temperature overnight and became unstirrable. 0.5 ml of Iso-butyl acetate was added and the suspension was stirred 20 hours at room temperature. The solid was then filtered from the suspension and analyzed by powder XRD.

### Example 23

Aliskiren base (50 mg) was dissolved in 0.5 ml of heptane-ethanol:10 volume-1 volume, and fumaric acid (5.3 mg) was then added. The suspension was stirred at room temperature overnight and became unstirrable. 0.5 ml of heptane:ethanol in a 10:1 volume ratio was added and the suspension was stirred 20 hours at room temperature. A sample of the solid from the suspension was then analyzed by powder XRD.

### Example 24

Aliskiren hemifumarate Form I (30 mg) was placed in an open beaker. The beaker was placed in a closed 125 ml vessel containing 20 ml of methyl tert-butyl ether, and kept at room temperature for 40 days. The obtained crystals were analyzed by powder XRD.

### Example 25

Aliskiren hemifumarate Form I (30 mg) was placed in an open beaker. The beaker was placed in a closed 125 ml vessel containing 20 ml of n-butyl acetate, and kept at room temperature for 40 days. The obtained crystals were analyzed by powder XRD.

### Example 26

Aliskiren hemifumarate (50 mg) was dissolved in 1.0 ml of butyl acetate by gradual heating (0.1-0.2° C. per minute) to 90° C. while stirring. The solution was cooled to 5° C. and then heated to room temperature. The solid was then filtered from the suspension and analyzed by powder XRD.

### Example 27

Aliskiren hemifumarate (100 mg) was dissolved in 0.25 ml of ethanol by stirring at room temperature. 1 ml of butyl acetate was added and the solution was stirred at room temperature for 30 hours giving suspension. The solid was then filtered from the suspension and analyzed by powder XRD.

### Preparation of Aliskiren Hemifumarate Form III

### Example 28

A suspension of aliskiren hemifumarate Form I (30 mg) was stirred in 0.3 ml of tetrahydrofuran at room temperature for 40 hours. The solid was then filtered from the suspension and analyzed by powder XRD. (See FIG. 3)

### Example 29

A suspension of aliskiren hemifumarate Form I (30 mg) was stirred in 0.3 ml of methyl acetate at room temperature for 40 hours. The solid was then filtered from the suspension and analyzed by powder XRD.

### Example 30

Aliskiren hemifumarate Form I (75 mg) was stirred in 0.75 ml of 2-butanol at room temperature for 40 hours. A sample of the solid from the suspension was analyzed by powder XRD.

### Preparation of Aliskiren Hemifumarate Form V

### Example 31

A suspension of aliskiren hemifumarate Form I (75 mg) in 0.75 ml of acetonitrile was stirred at room temperature for 40 hours. A sample of the solid from the suspension was analyzed by powder XRD and found to be Form V.

### Example 32

Aliskiren base (50 mg) was dissolved in 1 ml of acetonitrile, and fumaric acid (5.3 mg) was then added. The suspension was stirred for 40 hours at room temperature. The solid was then filtered from the suspension and analyzed by powder XRD.

### Example 33

Aliskiren hemifumarate Form I (50 mg) was dissolved in 1 ml of acetonitrile while gradual heating (0.1-0.2° C. per minute) to 78° C. while stirring. The solution was cooled slowly to 5° C. and then heated to room temperature. The resulting clear solution was kept at room temperature for 3 days, during which time it became a suspension. The solid was filtered from the suspension and analyzed by powder XRD.

### Example 34

Aliskiren hemifumarate amorphous Form (50 mg) was stirred in 0.5 ml of acetonitrile at room temperature for 40 hours. Acetonitrile (0.5 ml) was added twice to get a suspension which could be stirred. The solid was filtered from the suspension and analyzed by powder XRD.

### Example 35

Aliskiren hemifumarate (200 mg) was dissolved in 0.5 ml of ethanol. Acetonitrile (4.5 ml) was added and the mixture was heated to reflux and then cooled and kept at room temperature without stirring overnight. Then the solution was kept without stirring at room temperature for 3 days. The solid was filtered and analyzed by XRD.

### Preparation of Aliskiren Hemifumarate Form VII

### Example 36

Aliskiren hemifumarate (50 mg) was dissolved in tert-butanol (1 ml) by gradual heating (0.1-0.2° C. per minute) to 80° C. while stirring. The solution was cooled slowly to 5° C. and then heated to room temperature. A sample of the solid from the suspension was analyzed by powder XRD and found to be Form VII. (See FIG. 7)

### Preparation of Aliskiren Hemifumarate Form VIII

### Example 37

A suspension of Amorphous aliskiren hemifumarate (50 mg) in acetonitrile (0.5 ml) was stirred at room temperature for 40 hours. The solid was filtered from the suspension, dried at 50° C. under vacuum, analyzed by powder XRD and found to be Form VIII. (See FIG. 8)

### Example 38

Aliskiren hemifumarate (30 mg) was dissolved in 0.5 ml of ethanol. An open beaker with the solution was placed in a closed 125 ml vessel containing 20 ml of acetonitrile, and was kept at room temperature for 40 days. The obtained crystals were analyzed by powder XRD.

### Example 39

Aliskiren hemifumarate Form I (30 mg) was placed in an open beaker. The beaker was placed in a closed 125 ml vessel containing 20 ml of acetonitrile, and kept at room temperature for 40 days. The obtained crystals were analyzed by powder XRD.

### Example 40

Aliskiren hemifumarate Form VIII was prepared by drying aliskiren hemifumarate Form V at 50° C. under vacuum overnight.

### Example 41

Aliskiren hemifumarate Form I (100 mg) was stirred in 2 ml of acetonitrile at room temperature for 30 hours. The obtained solid was analyzed by powder XRD.

### Preparation of Aliskiren Hemifumarate Form IX

### Example 42

Aliskiren base (50 mg) was dissolved in 1 ml of diethylcarbonate, and fumaric acid (5.3 mg) was then added. The suspension was stirred for 40 hours at room temperature. The solid was then filtered from the suspension and analyzed by powder XRD.

### Example 43

Aliskiren base (50 mg) was dissolved in 1 ml of ethyl acetate and fumaric acid (5.3 mg) was added. The suspension was stirred for 40 hours at room temperature. The solid was then filtered from the suspension and analyzed by powder XRD. (See FIG. 9.)

### Example 44

Aliskiren hemifumarate (100 mg) was dissolved in 0.25 ml 1-propanol by stirring at room temperature. 1 ml of methyl tert-butyl ether was added and the solution was stirred at room temperature for 30 hours to give a suspension. The solid was then filtered from the suspension and analyzed by powder XRD.

### Preparation of Aliskiren Hemifumarate Form X

### Example 45

Aliskiren hemifumarate Amorphous Form (100 mg) was dissolved in 2.75 ml of isopropanol. The solution became a slurry after stirring at room temperature for 4 days. The solid was then filtered from the slurry and analyzed by powder XRD. (See FIG. 10.)

### Example 46

Aliskiren hemifumarate Amorphous Form (100 mg) was dissolved in 1 ml of isopropanol. The solution became a slurry after stirring at room temperature for 30 hours. The solid was the filtered from the slurry and analyzed by powder XRD.

### Preparation of Amorphous Aliskiren Hemifumarate

### Example 47

Aliskiren hemifumarate (50 mg) was dissolved in ethanol (5 ml) or methanol (5 mL). The ethanol or methanol was then evaporated to dryness under vacuum. The resulting solid was analyzed by powder XRD and found to be amorphous form of aliskiren hemifumarate.

### Example 48

A suspension of aliskiren hemifumarate Form I (30 mg) in 0.3 ml of iso-butanol was stirred at room temperature for 40 hours. A sample of the solid from the suspension was analyzed by powder XRD and found to be amorphous form of aliskiren hemifumarate.

### Example 49

A suspension of aliskiren hemifumarate Form I (30 mg) in 0.3 ml of amyl alcohol was stirred at room temperature for 40 hours. A sample of the solid from the suspension was analyzed by powder XRD and found to be amorphous form of aliskiren hemifumarate.

### Example 50

A suspension of aliskiren hemifumarate Form I (30 mg) in 0.3 ml of n-butanol was stirred at room temperature for 40 hours. A sample of the solid from the suspension was analyzed by powder XRD and found to be amorphous form of aliskiren hemifumarate.

### Example 51

A suspension of aliskiren hemifumarate Form I (30 mg) in 0.3 ml of tert-butanol was stirred at room temperature for 40 hours. The solid was filtered and dried at 50° C. under vacuum overnight and then analyzed by powder XRD. (See FIG. 8)

### Example 52

A suspension of aliskiren hemifumarate Form I (30 mg) in 0.3 ml of heptane was stirred at room temperature for 40 hours. The solid was filtered from the suspension and analyzed by powder XRD.

### Example 53

A suspension of aliskiren hemifumarate Form I (75 mg) in 0.75 ml of iso-propanol was stirred at room temperature for 40 hours. The solid was filtered from the suspension and analyzed by powder XRD.

### Example 54

A suspension of aliskiren hemifumarate Form I (75 mg) in 0.75 ml of tert-butanol was stirred at room temperature for 40 hours. A sample of the solid from suspension was analyzed by powder XRD.

### Example 55

Aliskiren hemifumarate (50 mg) was dissolved in decane (1 ml) by gradual heating (0.1-0.2° C. per minute) to 90° C. while stirring. The solution was slowly cooled to 5° C. and then heated to room temperature. A sample of the solid from the suspension was analyzed by powder XRD.

### Example 56

Aliskiren hemifumarate (50 mg) was dissolved in butyl acetate (1 ml) by gradual heating (0.1-0.2° C. per minute) to 90° C. while stirring. The solution was slowly cooled to 5° C. and then heated to room temperature. A sample of the solid from the suspension was analyzed by powder XRD.

### Example 57

Aliskiren hemifumarate (50 mg) was dissolved in dibutyl ether (0.75 ml) by gradual heating (0.1-0.2° C. per minute) to 90° C. while stirring. The solution was slowly cooled to 5° C. and then heated to room temperature. A sample of the solid from the suspension was analyzed by powder XRD.

### Example 58

Aliskiren hemifumarate (50 mg) was dissolved in dioxane (0.75 ml) by gradual heating (0.1-0.2° C. per minute) with stirring at 90° C. The solution was slowly cooled to 5° C. and then heated to room temperature. A sample of the solid from the suspension was analyzed by powder XRD.

### Example 59

Aliskiren hemifumarate (50 mg) was dissolved in dimethyl carbonate (1 ml) by gradual heating (0.1-0.2° C. per minute) with stirring at 90° C. The solution was slowly cooled to 5° C. and then heated to room temperature. The solid was filtered from the suspension and analyzed by powder XRD.

### Example 60

Aliskiren hemifumarate amorphous form was obtained from Form VII, (wet sample) by drying at 50° C. overnight under vacuum.

### Example 61

Aliskiren hemifumarate Form I (50 mg) was stirred in 0.5 ml of heptane containing 0.8 volume % of water at room temperature for 20 hours. The solid was filtered from the suspension and analyzed by powder XRD.

### Example 62

Aliskiren base (50 mg) was dissolved in 0.5 ml of MTBE, and fumaric acid (5.3 mg) was then added. The suspension was stirred at room temperature overnight and became unstirrable. 0.5 ml of MTBE was added and the suspension was stirred 20 hours at room temperature. A sample of the solid from the suspension was analyzed by powder XRD.

### Example 63

Aliskiren hemifumarate Form I (200 mg) was dissolved in 7 ml of butyl acetate by heating with stirring at 90° C. for 2 hours. The solution was cooled to room temperature, kept at room temperature overnight and then kept at 0-5° C. for 6 hours. The resulting solid was analyzed by powder XRD and found to be amorphous form of aliskiren hemifumarate.

### Example 64

Aliskiren hemifumarate Form I (200 mg) was dissolved in 12 ml of ethyl acetate by heating with stirring at reflux for 2 hours. The solution was cooled to room temperature, kept at room temperature overnight and then kept at 0-5° C. for 6 hours. The resulting solid was analyzed by powder XRD and found to be amorphous form of aliskiren hemifumarate.

### Example 65

Aliskiren hemifumarate Form I (100 mg) was dissolved in 2 ml of isobutyl acetate by heating with stirring at 90° C. for 2 hours. The solution was cooled to room temperature, kept at room temperature overnight and then kept at 0-5° C. for 6 hours. The resulting solid was analyzed by powder XRD and found to be amorphous form of aliskiren hemifumarate.

### Example 66

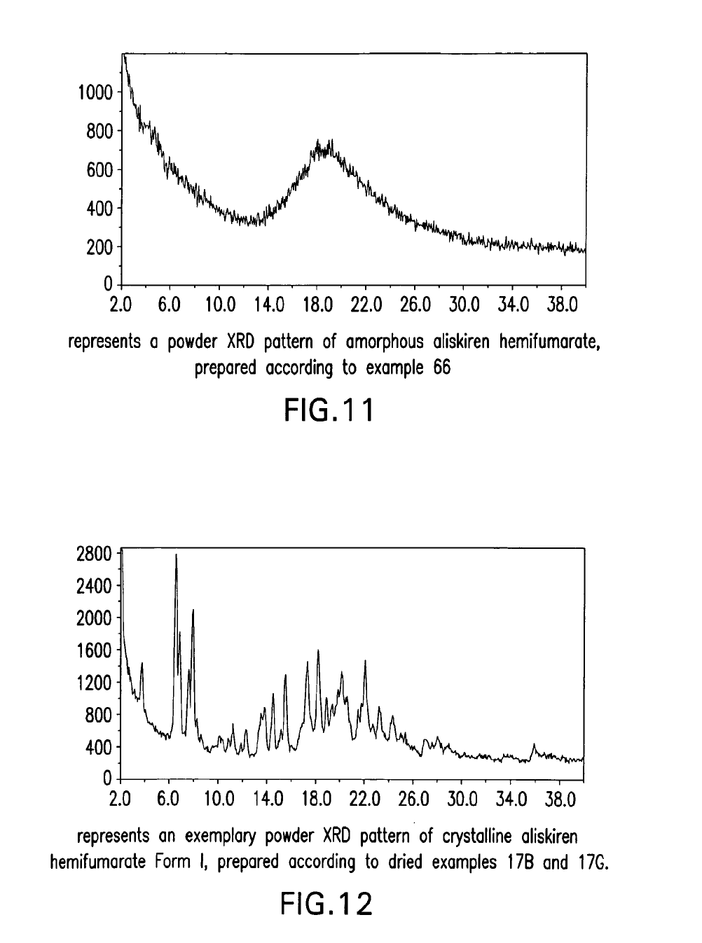

A suspension of aliskiren hemifumarate Form I (75 mg) in 0.75 ml of iso-butanol was stirred at room temperature for 40 hours. The solid was filtered from the suspension and analyzed by powder XRD and found to be amorphous form of aliskiren. (see FIG. 11)

### Example 67

Aliskiren base (5.5 g) was dissolved in 100 ml of ethanol, and fumaric acid (0.6 g) was then added. The suspension was stirred at room temperature and became a solution. The ethanol was then evaporated to dryness under vacuum. The resulting solid was analyzed by powder XRD and found to be amorphous form of aliskiren hemifumarate.

### Formulation of Amorphous Aliskiren Hemifumarate

### Example 67

The following formulation example of pharmaceutical preparation was prepared using amorphous Aliskiren hemifumarate as an active ingredient:

| component | amount (mg) | % weight |
|---|---|---|
| amorphous Aliskiren hemifumarate | 27.370 | 46.64 |
| microcrystalline cellulose | 17.738 | 30.23 |
| polyvinylpyrrolidone | 3.946 | 6.72 |
| crosspovidone | 7.876 | 13.42 |
| aerosil 200 | 0.300 | 0.51 |
| magnesium stearate | 1.454 | 2.48 |
| total | 58.684 | 100 |

The formulation was mixed and pressed to a tablet. The applied pressure was 2 tons for 1 minute.

The formulation was analyzed by XRPD and was found to contain amorphous Aliskiren hemifumarate. No conversion of the amorphous Aliskiren to a crystalline phase was observed.

### Example 68

The following formulation example of pharmaceutical preparation is prepared using amorphous Aliskiren hemifumarate as an active ingredient:

| component | amount (mg) | % weight |
|---|---|---|
| amorphous Aliskiren hemifumarate | 82.875 | 48.75 |
| lactose monohydrate | 54.625 | 32.13 |
| polyvinylpyrrolidone | 6.000 | 3.53 |
| crosspovidone | 24.000 | 14.12 |
| magnesium stearate | 2.500 | 1.47 |
| total | 170 | 100 |

The formulation is mixed and pressed to a tablet. The applied pressure is 2 tons for 1 minute.

The formulation is analyzed by XRPD and found to contain amorphous Aliskiren hemifumarate. No conversion of the amorphous Aliskiren to a crystalline phase is observed.

1. A process of preparing aliskiren hemifumarate (Form I) characterized by data selected from a group consisting of: a powder XRD pattern with peaks at about 3.8, 6.6, 7.6, 8.0, 13.8, 14.5, 15.6 and 17.4±0.2 degrees 2-theta, a powder XRD pattern with peaks at about 3.8, 7.6, 8.0, 13.8 and 15.6±0.2 degrees 2-theta with optional one or more additional peaks in the powder XRD pattern at about 6.6, 14.5 and 17.4±0.2 degrees 2-theta, a powder XRD pattern as depicted in FIG. 1, comprising: preparing a solution of aliskiren hemifumarate in a solvent selected from the group consisting of diethylcarbonate, tetrahydrofuran and ethyl acetate; and crystallizing the aliskiren hemifumarate.

2. The process according to claim 1, wherein the aliskiren hemifumarate is dissolved at a temperature between about 25° C. and about 100° C.

3. The process according to claim 1 or claim 2, wherein the crystallization is carried out by cooling the solution at a temperature of about −10° C. to about 20° C.

4. The process according to claim 1 or claim 2, wherein the solvent is ethyl acetate, and wherein a ratio of lower than about 1:40 g aliskiren hemifumarate to ml ethyl acetate is used (w/v).

5. The process according to claim 1 claim 2, wherein the solvent is ethyl acetate, and wherein a ratio of about 1:15 to about 1:25 of g aliskiren hemifumarate to ml ethyl acetate is used (w/v).

6. A process of preparing the aliskiren hemifumarate Form I of claim 1, comprising: drying aliskiren hemifumarate Form II, Form III, Form IX, Form X, or a mixture thereof to obtain the aliskiren hemifumarate Form I.

7. The process according to claim 6, wherein the drying step is carried out at about 25° C. to about 70° C.

8. A process of preparing the aliskiren hemifumarate Form I of claim 1, comprising: preparing a solution of aliskiren hemifumarate in a C₁ to C₃ alcohol, and combining with an antisolvent to crystallize the aliskiren hemifumarate Form I.

9. The process according to claim 8, comprising: dissolving an aliskiren base and fumaric acid in ethanol; and combining with acetonitrile.

10. The process according to claim 9, wherein the aliskiren hemifumarate/ethanol ratio used is 1:1 of g aliskiren hemifumarate to g ethanol (w/w) and the acetonitrile/ethanol volume ratio is 18:1 (v/v).

11. The process according to any one of claims 8 to 10, wherein the crystallization is carried out without seeding.

12. The process according to any one of claims 8 to 10, wherein the C₁ to C₃ alcohol is ethanol or 1-propanol.

13. The process according to any one of claims 8 to 10, wherein the antisolvent is selected from the group consisting of acetonitrile, isopropyl acetate, ethyl acetate, butyl acetate, isobutyl acetate, and methyl tert-butyl ether.

14. The process according to claim 13, wherein the antisolvent is butyl acetate, and wherein a ratio greater than about 1:15 of g aliskiren hemifumarate to ml butyl acetate is used (w/v).

15. The process according to claim 14, wherein the antisolvent is butyl acetate, and wherein a ratio of about 1:20 to about 1:30 of g aliskiren hemifumarate to ml butyl acetate is used (w/v).

16. The process according to claim 13, wherein the antisolvent is ethyl acetate, and wherein a ratio of lower than about 1:27 of g aliskiren hemifumarate to ml ethyl acetate is used (w/v).

17. The process according to claim 16, wherein the antisolvent is ethyl acetate, and wherein a ratio of about 1:15 to about 1:25 of g aliskiren hemifumarate to ml ethyl acetate is used (w/v).

18. The process according to claim 13, wherein the antisolvent is heptane.

19. A crystalline form of aliskiren hemifumarate (designated Form II), characterized by a powder XRD pattern with peaks at about 4.9, 7.3, 10.0 and 12.2±0.2 degrees 2-theta.

20. The aliskiren hemifumarate Form II according to claim 19, which is further characterized by one or more additional peaks in the powder XRD pattern at about 8.5, 9.5, 11.8 or 21.4±0.2 degrees 2-theta or a powder XRD pattern as depicted in FIG. 2.

21. The aliskiren hemifumarate Form II according to claim 19 or claim 20, which is further characterized by a powder XRD pattern with the absence of peak at about 9.2±0.2 degrees 2-theta.

22. A process of preparing the aliskiren hemifumarate Form II of claim 19 or claim 20, comprising: preparing a solution of aliskiren hemifumarate in iso-butyl acetate or butyl acetate; and crystallizing the aliskiren hemifumarate Form II.

23. The process according to claim 22, wherein aliskiren hemifumarate is dissolved at a temperature between about 25° C. and about 100° C.

24. The process according to claim 22 or 23, wherein the crystallization is carried out by cooling the solution at a temperature of about −10° C. to about 20° C.

25. A process of preparing the aliskiren hemifumarate Form II of claim 19 or claim 20, comprising: preparing a heterogeneous mixture of aliskiren hemifumarate Form I in an organic solvent selected from the group consisting of 2-pentanol, methyl tert-butyl ether, diethylcarbonate, butyl acetate, isobutyl acetate, ethanol, toluene, isopropanol, and mixtures thereof; and maintaining the mixture to obtain the aliskiren hemifumarate Form II.

26. The process according to claim 25, wherein the solvent is in a mixture with water.

27. The process according to claim 25 or claim 26, wherein the mixture is maintained for about 10 to about 40 hours.

28. A process of preparing the aliskiren hemifumarate Faun II of claim 19 or claim 20, comprising: dissolving an aliskiren base in iso-butyl acetate or heptane-ethanol; adding fumaric acid to form a heterogeneous mixture; and maintaining the mixture to obtain the aliskiren hemifumarate Form II.

29. A process of preparing the aliskiren hemifumarate Faun II of claim 19 or claim 20, comprising: preparing a solution of aliskiren hemifumarate in ethanol (solvent), optionally in mixture with water; and combining with an antisolvent to obtain the aliskiren hemifumarate Form II.

30. The process according to claim 29, wherein the antisolvent is selected from the group consisting of diethylcarbonate, ethyl acetate, and butyl acetate.

31. The process according to claim 30, wherein the antisolvent is ethyl acetate, and wherein a ratio of higher than about 1:27 of g aliskiren hemifumarate to ml ethyl acetate is used (w/v) and the solvent to anti-solvent ratio is about 1:12 to about 1:18 (v:v).

32. The process according to claim 30, wherein the antisolvent is butyl-acetate, and wherein a ratio of lower than about 1:15 g aliskiren hemifumarate to ml butyl acetate is used (w/v) and the solvent to anti-solvent ratio is about 1:3 to about 1:8 (v/v).

33. A process of preparing the aliskiren hemifumarate Form II of claim 19 or claim 20, comprising: preparing a mixture of amorphous aliskiren hemifumarate in dimethyl carbonate or isobutyl acetate; and maintaining the mixture to obtain the aliskiren hemifumarate Form II.

34. A process of preparing the aliskiren hemifumarate Form II of claim 19 or claim 20, comprising: placing aliskiren hemifumarate Form I in an open container; and maintaining the open container in a closed container containing a C₂-C₅ ether or a C₂-C₆ ester to obtain the aliskiren hemifumarate Form II.

35. A crystalline form of aliskiren hemifumarate (designated Form III), characterized by a powder XRD pattern with peaks at about 6.5, 7.4, 19.5 and 20.6±0.2 degrees 2-theta.

36. The aliskiren hemifumarate Form III according to claim 35, which is further characterized by one or more additional peaks in the powder XRD pattern at about 18.4 or 22.7±0.2 degrees 2-theta or a powder XRD pattern as depicted in FIG. 3.

37. The aliskiren hemifumarate Form III according to claim 35 or claim 36, which is further characterized by a powder XRD pattern with an absence of peak at about 8.0±0.2 degrees 2-theta.

38. A process of preparing the aliskiren hemifumarate Form III of claim 35 or claim 36, comprising: preparing a heterogeneous mixture of aliskiren hemifumarate Form I in a solvent selected from the group consisting of 2-butanol and methyl acetate; and maintaining the mixture to obtain the aliskiren hemifumarate Form III.

39. The process according to claim 38, wherein the ratio of aliskiren hemifumarate to the solvent is about 1:5 to about 1:15 of g aliskiren hemifumarate to ml of solvent (w/v).

40. A process of preparing the aliskiren hemifumarate Form III of claim 35 or claim 36, comprising: preparing a heterogeneous mixture of aliskiren hemifumarate Form I in tetrahydrofuran; and maintaining the mixture to obtain the aliskiren hemifumarate Form III.

41. A crystalline form of aliskiren hemifumarate (designated Form V), characterized by a powder XRD pattern with peaks at about 4.5, 7.0, 13.6 and 19.6±0.2 degrees 2-theta.

42. The aliskiren hemifumarate Form V according to claim 41, which is further characterized by one or more additional peaks in the powder XRD pattern at about 5.8, 18.2 or 22.8±0.2 degrees 2-theta or a powder XRD pattern as depicted in FIG. 4.

43. A process of preparing the aliskiren hemifumarate Form V of claim 41 or claim 42, comprising: preparing a solution of aliskiren hemifumarate in acetonitrile; and cooling the solution to obtain the aliskiren hemifumarate Form V.

44. The process according to claim 43, wherein the ratio of aliskiren hemifumarate to the solvent is about 1:5 to about 1:25 of g aliskiren hemifumarate to ml of solvent (w/v).

45. The process according to claim 43, wherein the ratio of aliskiren hemifumarate to the solvent is about 1:10 to about 1:20 of g aliskiren hemifumarate to ml of solvent (w/v).

46. A process of preparing the aliskiren hemifumarate Form V of claim 41 or claim 42, comprising: preparing a heterogeneous mixture of aliskiren hemifumarate Form I in acetonitrile; and maintaining the mixture to obtain the aliskiren hemifumarate Form V.

47. The process according to claim 46, wherein a ratio of aliskiren hemifumarate to acetonitrile is lower than about 1:15 g of aliskiren hemifumarate to ml of acetonitrile (w/v).

48. A process of preparing the aliskiren hemifumarate Form V of claim 41 or claim 42, comprising: preparing a heterogeneous mixture of amorphous aliskiren hemifumarate in acetonitrile; and maintaining the mixture to obtain the aliskiren hemifumarate Form V.

49. The process according to claim 48, wherein a ratio of aliskiren hemifumarate to acetonitrile is greater than about 1:15 g of aliskiren hemifumarate to ml of acetonitrile (w/v).

50. A process of preparing the aliskiren hemifumarate Form V of claim 41 or claim 42, comprising: dissolving an aliskiren base in acetonitrile; combining with fumaric acid to obtain a heterogeneous mixture; and maintaining the mixture to obtain the aliskiren hemifumarate Form V.

51. A process of preparing the aliskiren hemifumarate Form V of claim 41 or claim 42, comprising: preparing a solution of aliskiren hemifumarate in ethanol, adding acetonitrile to obtain a reaction mixture; heating the mixture to reflux; and then cooling the mixture to crystallize the aliskiren hemifumarate Form V.

52. A crystalline form of aliskiren hemifumarate (designated Form VII), characterized by a powder XRD pattern with peaks at about 4.3, 6.2, 12.4 and 18.8±0.2 degrees 2-theta.

53. The aliskiren hemifumarate Form VII according to claim 52, which is further characterized by one or more additional peaks in the powder XRD pattern at about 8.9, 10.0, 17.9 or 19.5±0.2 degrees 2-theta or a powder XRD pattern as depicted in FIG. 5.

54. The aliskiren hemifumarate Form VII according to claim 52 or claim 53, which is further characterized by a powder XRD pattern with peaks at about 4.3, 6.2, 12.4 and 18.8±0.2 and an absence of peak at about 7.1±0.2 degrees 2-theta.

55. A process of preparing the aliskiren hemifumarate Form VII of claim 52 or claim 53, comprising: preparing a solution of aliskiren hemifumarate in a C₁-C₄ alcohol; and cooling the solution to crystallize the aliskiren hemifumarate Form VII.

56. The process according to claim 55, wherein the C₁-C₄ alcohol is tert-butanol.

57. A process of preparing the aliskiren hemifumarate (Form VIII) characterized by a powder XRD pattern with peaks at about 6.0, 7.4, 9.3 and 11.1±0.2 degrees 2-theta., which is further characterized by one or more additional peaks in the powder XRD pattern at about 8.6, 10.0, 19.2, 19.7 or 20.1±0.2 degrees 2-theta or a powder XRD pattern as depicted in FIG. 6 comprising: preparing a heterogeneous mixture of acetonitrile and aliskiren hemifumarate Form I and maintaining the mixture to obtain the aliskiren hemifumarate.

58. The process of claim 57, wherein the ratio of aliskiren hemifumarate to acetonitrile is greater than about 1:15 g of aliskiren hemifumarate to ml of acetonitrile (w/v).

59. A process of preparing the aliskiren hemifumarate Form VIII of claim 57, comprising: preparing a heterogeneous mixture of acetonitrile and amorphous aliskiren hemifumarate and maintaining the mixture to obtain the aliskiren hemifumarate Form VIII.

60. The process of claim 59, wherein the ratio of aliskiren hemifumarate to acetonitrile is less than about 1:15 g of aliskiren hemifumarate to ml of acetonitrile (w/v).

61. A process of preparing the aliskiren hemifumarate Form VIII of claim 57, comprising maintaining an open container with aliskiren hemifumarate inside in a closed container containing acetonitrile to obtain the aliskiren hemifumarate Form VIII.

62. The process of claim 61, wherein the open container contains a solution of aliskiren hemifumarate in ethanol.

63. A process of preparing the aliskiren hemifumarate Form VIII of claim 57, comprising drying hemifumarate Form V to obtain the aliskiren hemifumarate Form VIII.

64. A crystalline form of aliskiren hemifumarate (designated Form IX) characterized by a powder XRD pattern with peaks at about 3.6, 6.5, 7.2, 12.4 and 18.0±0.2 degrees 2-theta.

65. The aliskiren hemifumarate Form IX according to claim 64, which is further characterized by one or more additional peaks in the powder XRD pattern at about 6.2, 8.5, 13.5, 18.9 or 22.3±0.2 degrees 2-theta or a powder XRD pattern as depicted in FIG. 7.

66. The aliskiren hemifumarate Form IX according to claim 64 or claim 65, which is further characterized by a powder XRD pattern with an absence of peak at about 8.0±0.2 degrees 2-theta.

67. A process of preparing the aliskiren hemifumarate Form IX of claim 64 or claim 65, comprising: preparing a solution of an aliskiren base in diethylcarbonate or ethyl acetate; adding fumaric acid to form a suspension; and maintaining the suspension to obtain the aliskiren hemifumarate Form IX.

68. A process of preparing the aliskiren hemifumarate Form IX of claim 64 or claim 65, comprising: preparing a solution of aliskiren hemifumarate in a C₁-C₃ alcohol; combining with methyl tert-butyl ether to obtain a reaction mixture; and maintaining the mixture to obtain the aliskiren hemifumarate Form IX.

69. A crystalline form of aliskiren hemifumarate (designated Form X) characterized by a powder XRD pattern with peaks at about 4.7, 6.3, 10.5 and 19.2±0.2 degrees 2-theta.

70. The aliskiren hemifumarate Form X according to claim 69, which is further characterized by one or more additional peaks in the powder XRD pattern at about 8.4, 9.6, 16.9, 19.5 or 24.2±0.2 degrees 2-theta or a powder XRD pattern as depicted in FIG. 8.

71. A process of preparing the aliskiren hemifumarate Form X of claim 69 or claim 70, comprising: preparing a solution of aliskiren hemifumarate in a C₁-C₃ alcohol; and maintaining the solution to obtain the aliskiren hemifumarate Form X.

72. A process of preparing an amorphous aliskiren hemifumarate comprising: preparing a solution of aliskiren hemifumarate in an organic solvent; and precipitating amorphous aliskiren hemifumarate from the solution.

73. The process of claim 72, wherein the aliskiren hemifumarate is dissolved at a temperature between 25° C. and 100° C.

74. The process of claim 72 or claim 73, wherein the precipitation is carried out by cooling the solution to a temperature between about 25° C. and about 0° C.

75. The process of claim 72 or claim 73, wherein the aliskiren hemifumarate is dissolved in butyl acetate, ethyl acetate, dimethyl carbonate, decane, dibutyl ether, iso-butanol and isobutyl acetate.

76. The process of claim 72 or claim 73, wherein the aliskiren hemifumarate is dissolved in dioxane.

77. The process of claim 72 or claim 73, wherein the organic solvent is ethyl acetate, and wherein a ratio greater than about 1:40 of g aliskiren hemifumarate to ml ethyl acetate is used (w/v).

78. The process of claim 72 or claim 73, wherein the organic solvent is ethyl acetate, and wherein a ratio of about 1:45 to about 1:65 of g aliskiren hemifumarate to ml ethyl acetate is used (w/v).

79. The process of claim 72 or claim 73, wherein the solution is prepared by dissolving aliskiren hemifumarate Form I in a C₂-C₆ ester.

80. A process of preparing an amorphous aliskiren hemifumarate comprising: preparing a heterogeneous mixture of aliskiren hemifumarate Form I and a solvent selected from the group consisting of a C₁-C₅ alcohol, a C₁-C₆ hydrocarbon and mixtures thereof; and maintaining the mixture to obtain the amorphous aliskiren hemifumarate.

81. The process according to claim 80, wherein the solvent is selected from the group consisting of isopropanol, iso-butanol, amyl alcohol, n-butanol, tert-butanol, heptane, and heptane-water.

82. A process of preparing an amorphous aliskiren hemifumarate comprising dissolving aliskiren base in methyl tert-butyl ether or ethanol; and
then combining with fumaric acid.

83. A process of preparing an amorphous aliskiren hemifumarate comprising: preparing a solution of aliskiren hemifumarate in a C₁-C₆ alcohol, and removing the alcohol from the solution to obtain the amorphous aliskiren hemifumarate.

84. A process of preparing an amorphous aliskiren hemifumarate comprising: drying aliskiren hemifumarate Form VII to obtain the amorphous aliskiren hemifumarate.

85. The process of any one of claims 72, 73, 80, 81, 82, 83 and 84, wherein the amorphous aliskiren hemifumarate is characterized by a powder XRD pattern as depicted in FIG. 9.

86. The process of any one of claims 72, 73, 80, 81, 82, 83 and 84, wherein the amorphous aliskiren hemifumarate has less than about 5 percent crystallinity.

87. The process of any one of claims 72, 73, 80, 81, 82, 83 and 84, wherein the amorphous aliskiren hemifumarate has less than about 3 percent crystallinity.

88. The process of any one of claims 72, 73, 80, 81, 82, 83 and 84, wherein the amorphous aliskiren hemifumarate is stable under a pressure of 2 tons for 1 minute.

89. Amorphous aliskiren hemifumarate that is stable under a pressure of 2 tons for 1 minute.

90. A pharmaceutical composition comprising an aliskiren hemifumarate polymorph selected from the group consisting of aliskiren hemifumarate Forms II, III, V, VII, IX, X, amorphous aliskiren hemifumarate stable under a pressure of 2 tons for 1 minute, and a pharmaceutically acceptable excipient.

91. A process of preparing the pharmaceutical composition of claim 90, comprising: preparing a mixture of the aliskiren hemifumarate polymorph with at least one pharmaceutically acceptable excipient; and processing the mixture to obtain the pharmaceutical composition.

92. A method of treating hypertension, comprising: administering to a subject the pharmaceutical composition of claim 90 in an amount effective for treating hypertension.

93. (canceled)

94. An aliskiren hemifumarate polymorph, made by a process according to any one of claims 1, 6, 8, 57, 59, 61, 63, 72, 80, 82, 83 and 84.
